(19) United States
(12) Patent Application Publication
(10) Pub. No.: US 2007/0287905 A1
(43) Pub. Date: Dec. 13, 2007

(54) Method for registering functional MR image data using radioscopy

(75) Inventor: Klaus Klingenbeck-Regn, Nurnberg (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 11/810,974

(22) Filed: Jun. 7, 2007

(30) Foreign Application Priority Data
Jun. 8, 2006 (DE) 10 2006 026 752.4

(51) Int. Cl. A61B 5/05

(52) U.S. Cl. 600/410

(57) ABSTRACT

The invention relates to a method and to a device for visualizing organs, in which a respective functional and anatomical magnetic resonance tomography image record of an organ are created, the anatomical magnetic resonance tomography image record containing visible landmarks and being registered with the functional magnetic resonance tomography image record. X-ray images of the organ are also taken which are then registered with the anatomical magnetic resonance tomography image record by using landmarks. The X-ray images and the images of the functional magnetic resonance tomography image record, which are registered with each other, can subsequently be displayed in a superimposed manner.

### CROSS REFERENCE TO RELATED APPLICATIONS

This application claims priority of German application No. 10 2006 026 752.4 filed Jun. 08, 2006, which is incorporated by reference herein in its entirety.

### FIELD OF THE INVENTION

The present invention relates to a method and to a device for visualizing organs. With the method and the device X-ray images are registered with functional magnetic resonance tomography images and visualized in a superimposed manner.

### BACKGROUND OF THE INVENTION

Functional magnetic resonance tomography (fMRT or fMRI (for functional magnetic resonance imaging)) is used in medical interventions to locate and display activated structures in internal organs, such as the brain, with high resolution.

Functional correlations of organs, such as the metabolic activity of areas of the brain, may be displayed thereby. What is known as the BOLD effect (blood oxygen level dependent) is used here, in that namely oxygenated and deoxygenated blood or hemoglobin exhibits different magnetic properties. Oxyhemoglobin is diamagnetic and does not affect the magnetic properties of tissue. Deoxyhemoglobin on the other hand is paramagnetic and this leads to discrete, but depictable changes in the magnetic field.

If for example areas of the cortex are activated or stimulated, increased metabolism occurs in the activated areas, so the activated area locally displays increased cerebral blood circulation. Consequently the ratio of oxygenated to deoxygenated hemoglobin changes. The effective cross relaxation time changes as a result and a signal change may be observed.

If images are successively taken in the normal state and in the activated state by means of functional magnetic resonance tomography, the activated areas of the organs can be located and visualized.

A conventional method of a functional magnetic resonance tomography comprises the following steps. Firstly what is known as a pre-scan is created, i.e. a brief scan with low resolution to check the position of a patient.

A three-dimensional magnetic resonance tomography scan with high resolution then takes place which visualizes the anatomy of the organ and the surrounding area for operation.

The actual functional magnetic resonance tomography scan then follows with low resolution and detects the activated areas of the organ. If for example the brain is being examined a stimulus is applied to one of the patient's nerves, such as to the foot or finger for example. During what is known as “finger tapping” the patient must move his finger toward the thumb. A stimulus is activated in the brain in the process. This stimulus is visible in the corresponding area of the brain in the magnetic resonance tomography scan in the form of colored markings.

In the case of minimal invasive interventions in the brain using needles, catheters or other instruments, the functional centers of the brain (motive, visual cortex, etc.) must be protected from damage and injury. This may be achieved if it is possible to visualize these functional centers from magnetic resonance tomography image data with their surroundings.

The “DynaCT” method is described in the article by Siemens Medical Solutions in issue no. 2/2005 dated Mar. 9, 2005 of MED.LETTER der DeutscheMedizintechnik.de. CT-like sections can be produced with this application using angiographic C-arm X-ray systems. Here the C-arm is moved in a circle around the patient and a defined number of projected images is acquired. These projections are then reconstructed to form tomographs as in a CT scanner.

DE 199 20 872 A1 describes a method for registering MR images with CT images in which when evaluating a voxel for the level of similarity, it is not only the image value of that voxel which is considered but also those of neighboring voxels. The possibility of registering functional MRT images with CT images, i.e. of spatially allocating them to each other, is also mentioned.

### SUMMARY OF THE INVENTION

The object of the present invention is to provide a method and a device for visualizing organs in which the functional centers and their surroundings may clearly be seen.

This object is achieved by a method and by a device with the features of the claims. Advantageous developments are defined in the subclaims.

The fact underlying the invention is that functional magnetic resonance tomography image data usually does not have any anatomical landmarks which correlate with the anatomy from X-ray images and could therefore be used for registration. Registration of the X-ray images or the three-dimensional, CT-like records reconstructed therefrom with the anatomical magnetic resonance tomography image record solves this problem since bones or soft tissue can be seen in the X-ray images and the anatomical magnetic resonance tomography image record which can be registered with each other.

According to the present invention an anatomical magnetic resonance tomography image record and X-ray images of the organ are taken in addition to a functional magnetic resonance tomography image record of an organ.

A three-dimensional, CT-like record (DynaCT) is also created using the same device with which the X-ray images are created during the actual medical intervention. The CT-like record (DynaCT) can be easily registered with the anatomical magnetic resonance tomography image record, whereby the X-ray images are also automatically registered with the anatomical magnetic resonance tomography image record since the X-ray images are created using the same unit with which the CT-like record was created. Since the anatomical magnetic resonance tomography image record is always registered with the functional magnetic resonance tomography image record the X-ray images are thereby advantageously also registered with the functional magnetic resonance tomography image record.

### BRIEF DESCRIPTION OF THE DRAWINGS

A preferred exemplary embodiment of the invention will now be described with reference to the accompanying drawings, in which:

FIG. 1 shows a schematized functional magnetic resonance tomography image record of a brain with an activated area;

FIG. 2 shows a schematized anatomical magnetic resonance tomography image record of a cranium;

FIG. 3 shows a superimposition of the functional and anatomical magnetic resonance tomography image records of FIGS. 1 and 2;

FIG. 4 shows a schematized X-ray image of the brain and the cranium;

FIG. 5 shows a schematic diagram of registration of the X-ray images with the anatomical magnetic resonance tomography image record and a superimposed depiction of the X-ray images and the images of the functional magnetic resonance tomography image record according to present invention; and

FIG. 6 shows an apparatus for visualizing organs according to the present invention.

### DETAILED DESCRIPTION OF THE INVENTION

An exemplary embodiment of the present invention will be described hereinafter with reference to the drawings.

With a minimal invasive intervention the area for operation should on the one hand be checked in real time using X-ray images, i.e. using radioscopy, and on the other hand the functional centers from the magnetic resonance tomography image data should be registered or merged with the X-ray images.

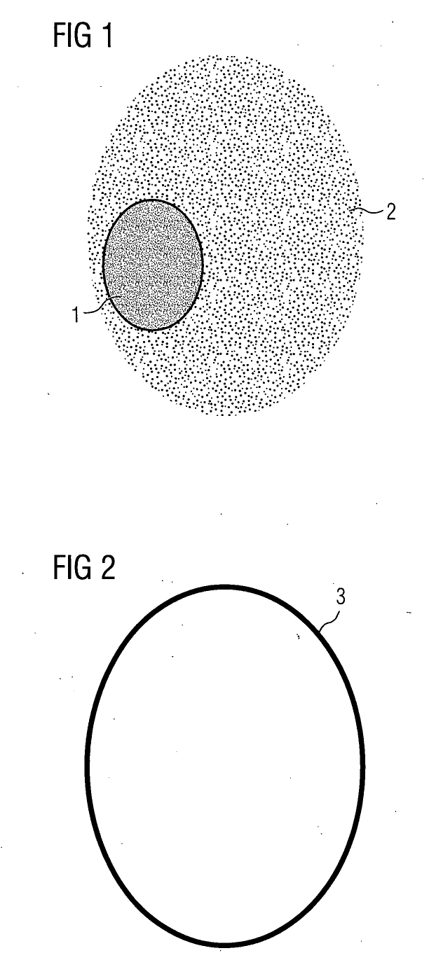

To achieve this, the first step lies in creating a functional magnetic resonance tomography image record of an organ, as is shown in FIG. 1. Reference numeral 2 schematically designates a brain that comprises an activated brain area 1.

In addition to the functional magnetic resonance tomography image record an anatomical magnetic resonance tomography image record of the organ is created, as is shown in FIG. 2. The anatomical magnetic resonance tomography image record contains visible landmarks in the form of a cranial bone 3. Other bones or soft tissue would also be suitable as landmarks even if this is not shown in the schematic diagram of FIG. 2.

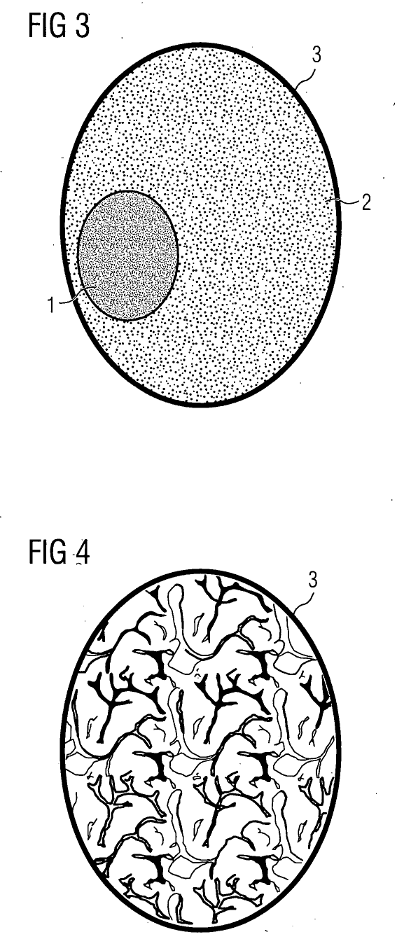

Since the patient virtually does not move between anatomical and functional magnetic resonance tomography scans, because he is positioned in a fixed head coil, the anatomical and functional magnetic resonance tomography scans can advantageously be registered with each other, as is shown in FIG. 3. The diagram in FIG. 3 contains the landmarks 3 and the activated region 1 of the brain 2.

The magnetic resonance tomography scans are pre-interventional images which are taken of the patient before the intervention and which are then available during the intervention (transfer via network, for example PACS).

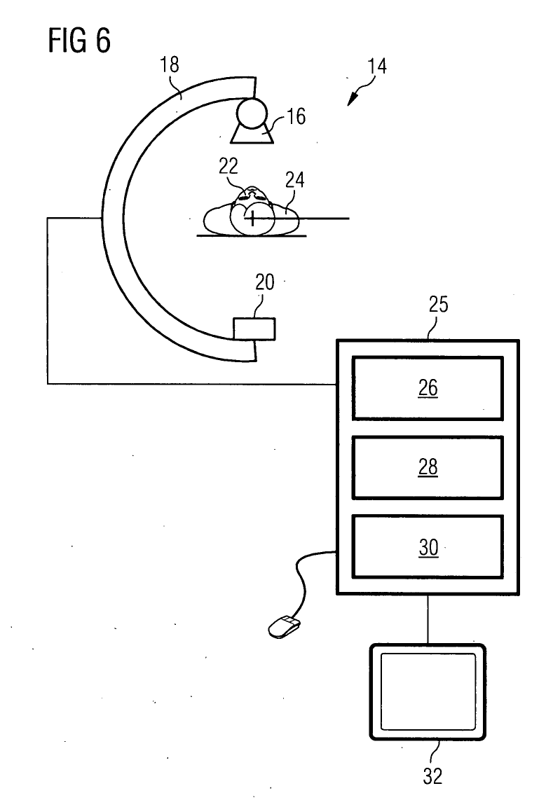

When the patient is finally subject to intervention three-dimensional, CT-like images of the anatomy (DynaCT) are first of all created using a rotating C-arm 18 of a C-arm X-ray device 14, as shown in FIG. 6. These CT-like images, like the anatomical magnetic resonance tomography scan, show the cranial bone 3 which is suitable as a landmark.

These transaxial tomographs of the CT-like images cover a three-dimensional volume which can be registered with the anatomical magnetic resonance tomography scan in FIG. 2 using the landmark 3. This can be done manually, semi-automatically or automatically.

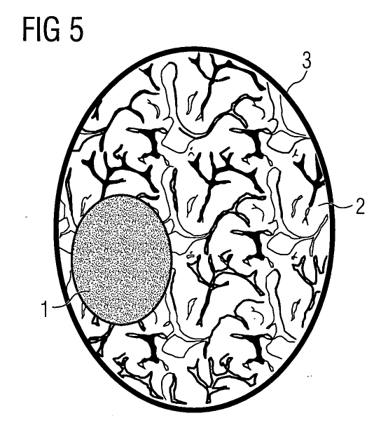

The actual X-ray images of the organ are then created preferably in real time during the intervention, as is shown in FIG. 4. If the X-ray images are created using the same unit 14 with which the three-dimensional, CT-like images of the anatomy (DynaCT) were created and if the patient does not move, the X-ray images created in real time are automatically registered with the CT-like images previously created. If the patient moves however, corrections may be necessary which compensate for the movement.

At the same time registering of the functional magnetic resonance tomography data with the X-ray images, i.e. with the X-ray anatomy of the patient, is achieved, since the functional magnetic resonance tomography image record and the X-ray images are registered with the CT-like images. FIG. 5 shows a superimposed depiction of the X-ray images and the images of the functional magnetic resonance tomography image record.

Use of interventional instruments can preferably be controlled in real time using the X-ray images. The instruments can thereby advantageously be guided in a targeted manner such that injury to functional centers in the brain can be avoided.

In addition the instruments can be equipped with a position sensor (medical GPS) which determines their position in the three-dimensional space. After appropriate calibration, position control of the instruments in the three-dimensional space of the anatomical and functional data can be carried out.

FIG. 6 shows a schematic diagram of a device for visualizing organs according to the present invention. The device has an apparatus 14 for taking DynaCT image data and X-ray images of the organ.

The apparatus 14 in this exemplary embodiment is an X-ray unit 14 with a connected device with which the fluoroscopic X-ray images are created. The X-ray device 14 is a C-arm device with a C-arm 18, on the arms of which an X-ray tube 16 and an X-ray detector 20 are provided. The device may for example be the Axiom Artis dFC belonging to Siemens AG, Medical Solutions, Erlangen, Germany. The patient 24 is located on a bed in the field of vision of the X-ray unit. Reference numeral 22 designates an organ inside the patient 24 which is the intended target of the intervention, such as the brain for example. A computer 25, which in the illustrated example controls the X-ray unit 14 and takes on the steps of registering the X-ray images with the anatomical magnetic resonance tomography image record and of depicting the images in a superimposed manner, is connected to the X-ray unit 14. These two functions can however also be implemented separately. In the illustrated example the C-arm movement and taking of intra-operative X-ray images is controlled by a control module 26.

FIG. 6 does not show the device for creating the functional and anatomical magnetic resonance tomography image record of the organ. This device is a conventional magnetic resonance tomography device however.

The pre-operatively taken functional and anatomical magnetic resonance tomography image records can be stored in a memory 28.

The X-ray images can be registered with the anatomical magnetic resonance tomography image record in a computing module 30 using landmarks 3. The X-ray images and the images of the functional magnetic resonance tomography image record can be displayed on a monitor 32 in a superimposed manner.

The computing module 30 is also capable of creating 3D reconstructions by means of DynaCT.

The present invention is not restricted to the illustrated embodiments; instead modifications are also incorporated by the scope of the invention which is defined by the accompanying claims.

1.-4. (canceled)

5. A method for visualizing an organ of a patient, comprising:
creating a functional magnetic resonance tomography image record of the organ;
creating an anatomical magnetic resonance tomography image record of the organ comprising a visible landmark and registered with the functional magnetic resonance tomography image record;
creating a three-dimensional CT-like image record of the organ comprising the visible landmark by a C-arm X-ray device;
registering the three-dimensional CT-like image record with the anatomical magnetic resonance tomography image record based on the visible landmark;
recording an X-ray image of the organ by the same C-arm X-ray device so that the X-ray image is also registered with the anatomical magnetic resonance tomography image record and therefore registered with the functional magnetic resonance tomography image record; and
superimposing the X-ray image with an image of the functional magnetic resonance tomography image record for visualizing the organ.

6. The method as claimed in claim 5, wherein a position of a medical instrument performing a medical procedure on the organ is detected by a position sensor and indicated in the superimposed X-ray image.

7. The method as claimed in claim 5, wherein the X-ray image of the organ is recorded in real time during a medical procedure.

8. The method as claimed in claim 5, wherein the functional and anatomical magnetic resonance tomography image records are created before a medical procedure.

9. The method as claimed in claim 8, wherein the pre-operatively created functional and anatomical magnetic resonance tomography image records are stored in a memory.

10. The method as claimed in claim 5, wherein the visible landmark is a bone or soft tissue of the organ.

11. A device to be used in a medical procedure performed on an organ of a patient, comprising:
a magnetic resonance tomography image device that creates:
a functional magnetic resonance tomography image record of the organ, and
an anatomical magnetic resonance tomography image record of the organ comprising a visible landmark of the organ and registered with the functional magnetic resonance tomography image record;
a C-arm X-ray device that:
creates a three-dimensional CT-like image record of the organ, and
records an X-ray image of the organ; and
a computer that:
registers the three-dimensional CT-like image record with the anatomical magnetic resonance tomography image record based on the visible landmark so that the X-ray image is also registered with the anatomical magnetic resonance tomography image record and therefore registered with the functional magnetic resonance tomography image record, and
superimposes the X-ray image with an image of the functional magnetic resonance tomography image record.

12. The device as claimed in claim 11, further comprising a display device that displays the superimposed X-ray image.

13. The device as claimed in claim 11, wherein a position of a medical instrument performing the medical procedure on the organ is detected by a position sensor and indicated in the superimposed X-ray image.

14. The device as claimed in claim 11, wherein the X-ray image of the organ is recorded in real time during the medical procedure.

15. The device as claimed in claim 11, wherein the functional and anatomical magnetic resonance tomography image records are created before the medical procedure.

16. The device as claimed in claim 15, further comprising a memory that stores the pre-operatively created functional and anatomical magnetic resonance tomography image records.

17. The device as claimed in claim 9, wherein the visible landmark is a bone or soft tissue of the organ.
